(19) United States
(12) Patent Application Publication
(10) Pub. No.: US 2026/0191457 A1
(43) Pub. Date: Jul. 9, 2026

(54) SYSTEMS AND METHODS FOR ASSESSING BREAST HEALTH CONDITIONS

(71) Applicant: HEALTH 1951, Saint-Étienne (FR)

(72) Inventors: Marie-Valérie MORENO, Chasselay (FR); Aurélie Marie BEGLE, Tramoyes (FR); Colin GUTTON, Vernaison (FR); Jean-Philippe Maurice MASSARDIER, Saint-Étienne (FR)

(21) Appl. No.: 19/440,420

(22) Filed: Jan. 5, 2026

Related U.S. Application Data
(60) Provisional application No. 63/741,937, filed on Jan. 5, 2025.

(51) Int. Cl. A61B 5/00; A61B 5/01; A61B 5/0535; A61B 5/256

(52) U.S. Cl. CPC A61B 5/4312; A61B 5/01; A61B 5/0535; A61B 5/256; A61B 5/6804; A61B 5/7225; A61B 5/725; A61B 5/7275

(57) ABSTRACT

A wearable breast-assessment device integrated into a garment uses contact sensor arrays to acquire bioimpedance via quadripolar measurements and, in certain embodiments, temperature and PPG data. An analog front end injects alternating current through selected electrodes and senses differential voltage at other electrodes. A multiplexing network electronically routes drive and sense terminals to any electrodes to implement numerous electrode-pair combinations without moving the garment. Processors execute programmed sequences, including frequency sweeps, convert complex impedance to resistance (R) and reactance (X), perform contact validation and averaging, and determine parameters indicative of tissue condition. In some embodiments, the device generates (i) two-dimensional slice images and (ii) three-dimensional reconstructions from multiple slices via Electrical Impedance Tomography.

### CROSS-REFERENCE TO RELATED APPLICATION

This application claims the benefit of priority under 35 U.S.C. § 119(e) to prior U.S. Provisional Application No. 63/741,937 filed Jan. 5, 2025, the entire disclosure of which is incorporated herein by reference in its entirety.

### BACKGROUND

The identification and assessment of medical conditions are critical components of effective healthcare delivery. Accurate diagnosis and monitoring are essential for ensuring appropriate treatment and improving patient outcomes. However, the current methods for identifying and assessing certain medical conditions, particularly those related to breast health, often involve invasive procedures. These procedures can be associated with significant drawbacks, including the need to schedule invasive procedures, patient discomfort, risk of complications, and extended recovery times.

One of the primary challenges in breast health is the detection of breast tumors. Traditional methods such as biopsies, while effective, require surgical intervention to obtain tissue samples for histological analysis.

Another significant concern is the assessment of lymphedema, a condition characterized by the accumulation of lymphatic fluid, often occurring after breast cancer surgery. Lymphedema can lead to swelling, discomfort, and impaired mobility. Current assessment methods may involve visits to specialists to assess lymphedema, leading to delay in identification and posing additional risks to the patient.

Breast mastitis, an inflammatory condition of the breast tissue, also presents diagnostic challenges. While clinical examination and imaging techniques such as ultrasound are commonly used, these methods typically require a qualified physical to assess the condition, leading to similar drawbacks as those encountered with tumor detection and lymphedema detection.

Post-operative recovery of breast tissue following surgical interventions, such as mastectomy or reconstructive surgery, is another area where non-invasive assessment methods are needed. Monitoring the healing process and identifying complications such as infections or tissue necrosis typically require physical examinations and sometimes additional surgical interventions. These methods can be uncomfortable for the patient and may not always provide comprehensive insights into the recovery process.

In light of these challenges, there is a pressing need for non-surgical methods to identify and assess medical conditions related to breast health. Such methods would ideally provide accurate and timely information to guide clinical decision-making. Addressing these needs could significantly enhance patient care, improve outcomes, and reduce the overall burden on healthcare systems.

### SUMMARY

A system of one or more computers can be configured to perform particular operations or actions by virtue of having software, firmware, hardware, or a combination of them installed on the system that, in operation, causes the system to perform the actions. One or more computer programs can be configured to perform particular operations or actions by virtue of including instructions that, when executed by data processing apparatus, cause the apparatus to perform the actions.

In one aspect, a wearable medical device for assessing a condition of breast tissue of a subject is disclosed. The device comprises a garment configured to position a first sensor array over a first breast and a second sensor array over a second breast, each sensor array including a plurality of bioimpedance electrodes arranged on a flexible substrate and configured to contact skin of the corresponding breast, an analog front-end configured to perform quadripolar bioimpedance measurements by injecting an alternating current into the breast tissue via a selected pair of the bioimpedance electrodes and sensing a differential voltage via a different selected pair of the bioimpedance electrodes to obtain complex impedance samples comprising resistance and reactance values at one or more frequencies, a multiplexing network operatively coupled between the analog front-end and the plurality of bioimpedance electrodes and configured to selectively route, under electronic control, drive terminals of the analog front-end to any electrodes of the plurality of bioimpedance electrodes for current injection, and sense terminals of the analog front-end to any electrodes of the plurality of bioimpedance electrodes for voltage measurement, thereby implementing a plurality of distinct electrode-pair combinations, and one or more processors and a non-transitory memory storing instructions that, when executed, cause the device to control the multiplexing network to execute a programmed measurement sequence defining a set of quadripolar electrode-pair combinations to generate a set of bioimpedance measurements across different spatial paths through the breast tissue and determine, based on the resistance and reactance values, a parameter indicative of the breast tissue condition.

Implementations may include one or more of the following features. The device provides that the parameter comprises one or more of a spatially resolved image of impedance-related values over a two-dimensional slice of the breast generated from the set of bioimpedance measurements, a three-dimensional reconstruction obtained from a plurality of two-dimensional slices, and a diagnostic score corresponding to presence or progression of a pathology.

The device provides that the sensor array further comprises a plurality of temperature sensors configured to measure cutaneous temperature at multiple locations on the corresponding breast and the memory stores instructions, that when executed, cause the device to acquire temperature data from the plurality of temperature sensors and determine the parameter further based on the acquired temperature data.

The device provides that the sensor array further comprises one or more photoplethysmography (PPG) sensors, and the memory stores instructions, that when executed, cause the device to acquire PPG data from the one or more PPG sensors and determine the parameter further based on the acquired PPG data.

The device provides that the programmed measurement sequence comprises two or more frequencies.

The device provides that the programmed measurement sequence comprises a frequency sweep between approximately 4 kHz and approximately 128 kHz.

The device provides that implementing the plurality of distinct electrode-pair combinations is performed without physically moving the garment.

The device provides that the memory stores instructions, that when executed, cause the device to perform signal conditioning on the bioimpedance measurements including at least contact validation and averaging to produce stabilized resistance and reactance values per electrode-pair combination and per frequency, wherein the parameter indicative of the breast tissue condition is based on the stabilized resistance and reactance values.

The device provides that the one or more processors are further configured to perform a frequency-domain analysis comprising, for each of a plurality of discrete excitation frequencies within a sweep spanning from approximately 4 kHz to approximately 128 kHz, driving an alternating current through a selected drive electrode pair and demodulating a corresponding sensed voltage at a selected sense electrode pair to obtain complex impedance samples, converting the complex impedance samples into resistance (R) and reactance (X) values expressed in ohms for each frequency, and storing, for each electrode-pair combination, a multi-frequency vector of R and X values usable as inputs to downstream modeling.

The device provides that the frequency-domain analysis further comprises calculating, per frequency and per electrode-pair combination, one or more of impedance magnitude |Z|, impedance phase angle, and ratios or differences between low-frequency and high-frequency impedances, including a ratio of Z at a low frequency to Z at a high frequency.

The device provides that the one or more processors are further configured to fit a Cole-Cole model to the multi-frequency complex impedance data to estimate dispersion parameters comprising one or more of extracellular resistance (Re), intracellular resistance (Ri), infinite-frequency resistance (Roo), a dispersion shape factor (a), a characteristic time constant or frequency (r or fc), and an effective membrane capacitance (Cm).

The device provides that the one or more processors are configured to derive, from the fitted Cole-Cole model and the measured data, one or more features selected from phase angle at a specified frequency, a vector composed of normalized resistance and reactance (BIVA), an extracellular-to-intracellular resistance ratio, and at least one spectroscopic contrast metric formed from two or more frequencies.

The device provides that the one or more processors are configured to generate model inputs for a logistic classification model, the model inputs comprising one or more of resistance and reactance values, one or more Cole-Cole parameters, and temperature-derived metrics.

The device provides that the logistic classification model comprises a binary logistic regression configured to output a probability or risk score of a pathology, the binary logistic regression receiving as explanatory variables one or more of resistance at approximately 80 kHz, resistance and reactance at approximately 40 kHz, resistance and reactance at approximately 4 kHz, and temperature-derived features.

The device provides that the one or more processors are configured to standardize at least a subset of the model inputs by mean-centering or dividing by a standard deviation derived from a reference dataset or a patient-specific baseline to generate normalized features prior to logistic classification.

The device provides that converting complex impedance samples into resistance and reactance values comprises demodulating the sensed voltage at the excitation frequency, applying programmable bioimpedance-channel high-pass filters, low-pass filters, or both high-pass and low-pass filters, and averaging a plurality of successive samples to obtain stabilized R and X per frequency.

The device provides that the one or more processors are configured to reject bioimpedance data that fails a quality criterion including one or more of a bipolar contact impedance threshold, a quadripolar impedance range validity window, a current-generator compliance monitor threshold, and a digital over-/under-voltage lead-off threshold.

The device provides that the multiplexing network is configured to execute programmed sequences defining sector-based traversals and line-scan traversals, each traversal comprising a plurality of distinct electrode-pair combinations that create different sensitive current paths through the breast tissue, thereby producing beam data suitable for image reconstruction.

The device provides that generating the spatially resolved image over a two-dimensional slice comprises solving an Electrical Impedance Tomography (EIT) inverse problem.

The device provides that generating the three-dimensional reconstruction comprises stacking a plurality of two-dimensional slices reconstructed at different anatomical levels and orientations and interpolating between the levels to form a volumetric impedance map.

### BRIEF DESCRIPTION OF THE DRAWINGS

FIG. 1 shows an exemplary medical device, according to some implementations of the present disclosure.

FIG. 2 shows an exemplary sequence diagram that illustrates the signal flow in one quadripolar bioimpedance measurement, according to some implementations of the present disclosure.

FIG. 3 is a flowchart illustrating a method for assessing a condition of breast tissue of a subject, according to some implementations of the present disclosure.

FIG. 4 shows example results according to a method for determining the presence of a breast tumor, according to some implementations of the present disclosure.

FIG. 5 shows example results according to a method for determining the presence of a breast tumor, according to some implementations of the present disclosure.

FIG. 6 shows another exemplary medical device, according to some implementations of the present disclosure.

FIG. 7 shows another exemplary medical device, according to some implementations of the present disclosure.

### DETAILED DESCRIPTION

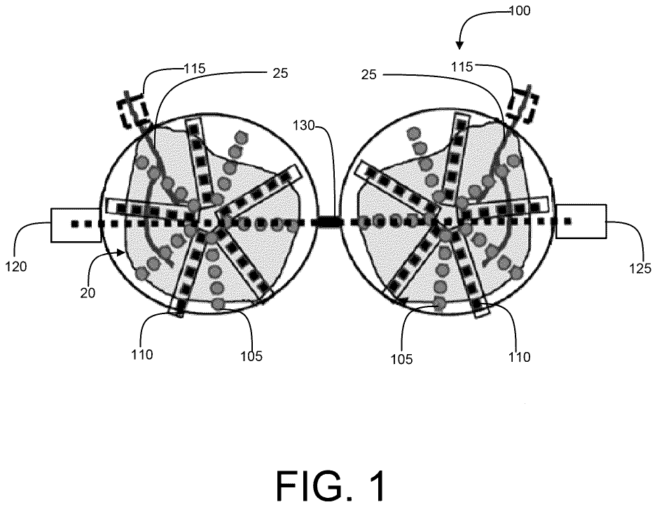

Referring to FIG. 1, a wearable medical device 100 is configured to conform to and overlie the breasts 20 of a subject and to acquire multi-parameter physiological data from the breast surface and underlying tissues. The device 100 includes an array of bioimpedance electrodes 105 distributed over each breast cup to contact the skin, a plurality of temperature sensors 110 positioned to measure cutaneous temperature across the breast surface, and one or more photoplethysmography (PPG) sensors 115 configured to optically interrogate superficial vasculature, including, in certain embodiments, an artery 25 supplying the breast, to obtain pulse and oxygenation-related signals for perfusion context. The sensors 105, 110, 115 are operatively coupled to a signal-conditioning and conversion chain comprising a multiplexer 120, an analog front end 125, and a microcontroller 130, arranged to support electronically reconfigurable quadripolar bioimpedance measurements, synchronous or staged acquisition of temperature and PPG data, and on-device preprocessing and control of programmed measurement sequences (e.g., frequency sweeps and electrode-pair selection). Although not shown in FIG. 1, in some implementations, medical device 100 includes additional temperature sensors 110 disposed to be in contact with a neutral reference site on the body, such as a location on the back of the user for temperature-based calibration and to mitigate inter-breast asymmetries due to vascularization or hormonal state. In some implementations, the neutral calibration value is acquired contemporaneously with breast measurements and used to tare temperature-dependent terms and to reduce bias when computing parameters indicative of tissue condition.

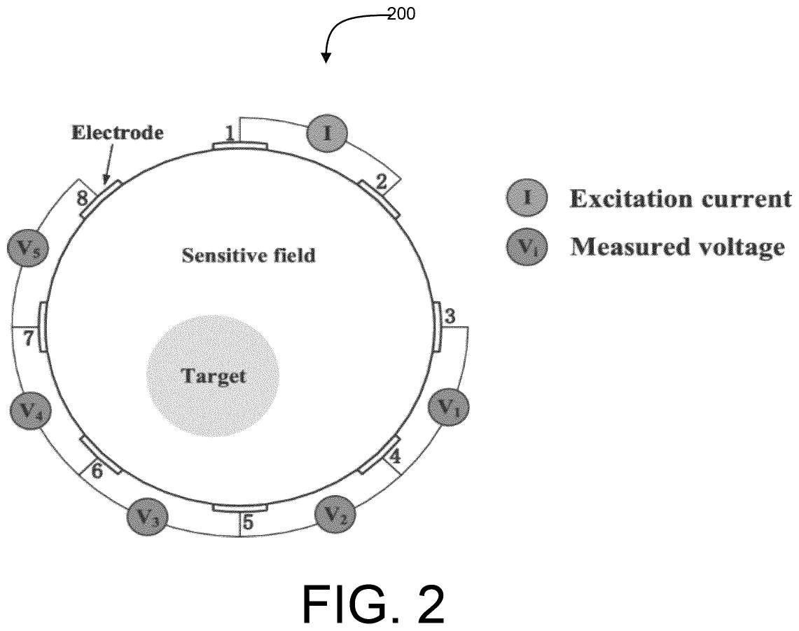

In some implementations, the bioimpedance electrodes 105 are organized on a flexible substrate so that selected electrodes can be designated as current-injection and voltage-sensing pairs under control of the multiplexer 120 to realize quadripolar measurements without physical movement of the device 100. In exemplary implementations, the electrode network supports numerous electrode-pair combinations spanning the breast surface to generate bioimpedance measurements along different spatial paths through the breast tissue as depicted schematically by excitation and measurement roles (I+/I−; V+/V−) and associated sensitive fields as described in more detail with respect to FIG. 2. The multiplexer 120 is configured to route, under electronic control, the drive terminals and sense terminals of the analog front end 125 to any selected electrodes 105 to establish the desired current injection and differential voltage sensing topology for each programmed measurement, enabling sector and line-scan traversals across the breast 20.

In some implementations, the analog front end 125 is implemented as a biopotential/bioimpedance AFE configured to generate a programmable alternating excitation current and to acquire demodulated bioimpedance samples. In operation, the analog front end 125 injects an AC current into the breast tissue via a selected pair of electrodes 105 and senses a differential voltage at a different selected pair of electrodes 105, producing complex impedance samples with resistance (R) and reactance (X) components at one or more frequencies. The AFE 125 includes input protection and filtering, a programmable current source, quadrature demodulation, and high-resolution conversion suitable for 2- or 4-electrode measurements, and supports lead-on/off and compliance monitoring to validate contact prior to acquisition.

In some implementations, the microcontroller 130 coordinates electrode addressing and measurement timing by issuing control signals to the multiplexer 120 and configuration commands to the analog front end 125, and by acquiring digitized data streams for local preprocessing and communication to higher-level processors. In exemplary embodiments, the microcontroller 130 manages multiple I2C buses to interrogate temperature sensors 110, controls SPI links to the AFE 125 and the multiplexer 120, and executes firmware that sequences frequency selection, averaging, and quality gating (e.g., contact validation thresholds) for each electrode-pair combination. In addition, the microcontroller 130 implements network connectivity to an external user device over a communications channel, such as via a wireless transceiver (e.g., Bluetooth Low Energy) and/or a wired interface (e.g., USB), and can communicate through a gateway to a cloud service. Over this channel, the microcontroller 130 is configured to (i) receive programmatic requests from an external user device—including, without limitation, requests to initiate or stop acquisitions, select frequencies, define or modify programmed measurement sequences, adjust electrode mapping, and request status or quality-control reports—and (ii) transmit, to the external user device, data packages containing the data collected by the medical device 100. Each transmitted data package may include, for example, timestamped complex impedance samples (R/X), associated temperature and/or PPG samples, electrode addressing metadata identifying the selected drive and sense electrodes, acquisition settings (frequency, gain, filter states), and quality indicators (lead/contact status, compliance and range checks), optionally formatted with framing, checksums, and encryption suitable for secure transport over the network.

In some implementations, the temperature sensors 110 are arranged in distributed positions over each breast cup to provide spatially resolved skin-temperature measurements contemporaneous with bioimpedance acquisitions, enabling computation of temperature gradients and correlation with impedance-derived parameters indicative of inflammation or other pathophysiology. The PPG sensors 115 are positioned to optically interrogate superficial microvascular and, where applicable, arterial blood flow at or near an artery 25 to provide pulse waveform and oxygenation context, which may be used to supplement bioimpedance-derived assessments of tissue state and perfusion.

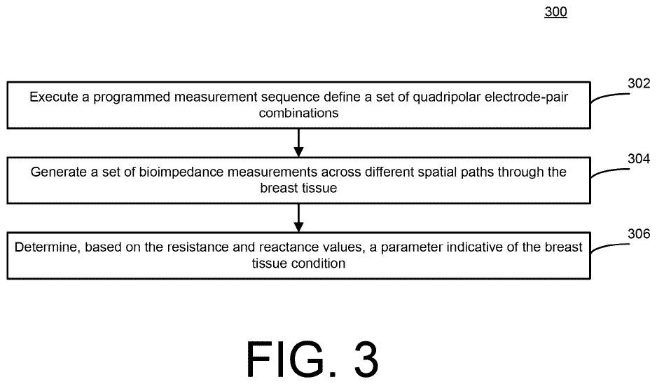

In use, and as described in more detail with respect to FIG. 3 below, the microcontroller 130 commands the multiplexer 120 to connect selected electrodes 105 to the analog front end 125 to establish a quadripolar configuration, instructs the analog front end 125 to drive an excitation current at a programmed frequency, and acquires the resulting complex impedance data. The device 100 thereby executes a programmed measurement sequence defining a set of electrode-pair combinations to generate bioimpedance measurements across different spatial paths through the breast tissue. The microcontroller 130 additionally acquires temperature data from the temperature sensors 110 and, in certain embodiments, PPG data from the PPG sensors 115, and conditions these signals (including contact validation and averaging) prior to further processing to determine parameters indicative of the condition of the breast tissue.

In some implementations, the medical device 100 can include 120 temperature sensors 110 (sixty per breast) that are organized into branches radiating outward from the center of each cup. In one example, each cup of the medical device includes 12 branches of five temperature sensors 110. It should be understood that other numbers of temperature measurement sensors, and other arrangements thereof are contemplated. In some implementations, the medical device can include 120 bioimpedance electrodes 105 (e.g., 60 per breast 20) organized into branches radiating outward form the center of each cup. In one example, each cup of the medical device includes 12 branches of five bioimpedance electrodes 105. In some implementations, the medical device can include one or more additional electrodes that may be used for general quadripolar measurements of breast tissue. In one example, the medical device includes up to four additional electrodes for quadripolar measurements of breast tissue. It should be understood that other numbers of electrodes, and other arrangements thereof are contemplated.

In some implementations, the electrode geometry can be configured either as a distributed array of individual bioimpedance electrodes 105 arranged in branches or, alternatively, as a small set of large circular electrodes (e.g., three or four large circular electrodes per breast 20) to simplify placement and reduce sequence complexity. In some implementations, the large circular electrode configuration enables a rapid acquisition cycle because only a limited number of electrode-pair combinations are required to obtain global or regional bioimpedance metrics. In some implementations, a full acquisition with large circular electrodes can be completed in seconds, subject to quality and safety checks. In some implementations, the multiplexer 120 and analog front end 125 are configured to support either geometry without mechanical changes to the garment.

The electrodes provided on the medical device are configurable to apply alternating currents to target tissues. In some implementations, a microcurrent of approximately 32 pA is applied to target tissue by the one or more electrodes. A range of microcurrents may be selected as desired for the analysis. In some implementations the microcurrent is selected in the range between approximately 8 μA and 100 μA.

The medical device can apply a range of different frequency alternating currents to the target tissues. According to some implementations, the electrodes of the medical device are configurable to provide alternating currents with frequencies in the range between approximately 4 kHz and 128 kHz. It should be noted that frequencies selected by the medical device are based on the type of information that can be collected about different areas of the target tissue. For example, a lower frequency (e.g., 4 kHz) may be useful to collect information on the extracellular environment. Higher frequencies (e.g., 128 kHz) are useful to collect information on the intracellular environment of the target tissue, and middle frequencies (e.g., between 4 kHz and 128 kHz) are useful to collect information on cell membranes in the target tissue. In some examples, one or more of the following frequencies are selected for use: approximately 4 kHz, 8 kHz, 18 kHz, 40 kHz, 80 kHz, and 128 kHz.

In some implementations, electrodes of the medical device are configurable to detect and quantify response signals received from the target tissue in response to the applied current. These response signals may be used by the one or more processor to perform bioimpedance spectroscopic analysis. In some implementations, bioimpedance spectroscopic analysis may include obtaining a parameter associated with the reactance of the response signal. In some implementations, bioimpedance spectroscopic analysis may include obtaining a parameter associated with the resistance of the response signal.

In some implementations, a memory stores instructions that, when executed by one or more processors coupled to the medical device, allows the medical device to assess one or more medical conditions based on data collected from one or more of the sensors. In one example, the medical device may apply alternating currents at various frequencies to obtain response signals that provide information corresponding to different internal structures of the target tissue. The medical device may perform a bioimpedance spectroscopic analysis on the response signal(s), and based on the analysis, and/or data additionally collected from the temperature sensor(s) and the PPG sensor(s), the device may determine a parameter corresponding to a medical condition.

In some implementations, the medical device 100 can detect the presence of a tumor. According to some implementations, the medical device detects the presence of a tumor based on estimating breast density through bioimpedance, collected temperature readings, and/or collected blood saturation levels across areas of the target tissue.

In some implementations, the medical device 100 can determine the level of inflammation and/or lymphedema in breast tissue post operative-recovery. According to some implementations, the medical device determines a level of inflammation in the breast tissue based on estimating breast density through bioimpedance, collected temperatures, and/or collected blood saturation levels across areas of the target tissue.

In some implementations, an auxiliary patch or armband positioned at the arm level (not shown in FIG. 1) is used to acquire additional bioimpedance or temperature measurements for applications such as lymphedema monitoring or perfusion context, and the medical device 100 may process these auxiliary measurements in conjunction with breast data.

In some implementations, the medical device 100 can detect the presence of mastitis in breast tissue. According to some implementations, the medical device determines the presence of mastitis in the breast tissue based on estimating breast density through bioimpedance, collected temperatures, and/or collected blood saturation levels across areas of the target tissue.

According to some examples, the medical device 100 can estimate breast tissue density using a multivariable linear regression model, as described by Equation (1) below:

$$Breast density = a + b {x}_{1} + c {x}_{2} + … + i {x}_{i} . \qquad ( 1 )$$

In Equation (1), a, b, . . . , i are constants and x₁, x₂, . . . , xᵢ are experimental variables of the equation. According to some examples, each of the data points collected by the medical device can be modeled as experimental variables of the model. According to some examples, the medical device 100 can further determine a parameter associated with the medical condition being assessed. For example, the medical device can define a parameter (e.g., a risk score) according to Equation (2) below:

$$Risk score = \frac{1}{( 1 + {e}^{( - ( a + b {x}_{1} + c {x}_{2} + … + i {x}_{i} ) )} )} . \qquad ( 2 )$$

In Equation (2), a, b, . . . , i are constants and x₁, x₂, . . . , xᵢ are experimental variables of the equation. In some implementations, depending on the number of return signals collected, equation (2) may be simplified. For example, a simplified logistic binomial law with two parameters (μ, s) may be used as shown in Equation (3) instead of Equation (2), where the two parameters may be associated with the received response signals:

$$f ( x ; μ , s ) = \frac{{e}^{- ( ( x - μ ) / s}}{{s ( 1 + {e}^{- ( \frac{x - μ}{s} )} )}^{2}} . \qquad ( 3 )$$

For equation (3), the distribution function may be calculated according to Equation (4):

$$f ( x ; μ , s ) = \frac{1}{1 + {e}^{- ( \frac{x - μ}{s} )}} . \qquad ( 4 )$$

When the expected value of E(x)=μ=0, and the variance Var(x)=s²π²/3, s can be set to 1 and the obtained distribution function can be described according to Equation (5):

$$f ( x ) = \frac{1}{1 + {e}^{- x}} , \qquad ( 5 )$$

wherein x corresponds to the electrical characteristics that are measured as part of the response signals. It should be understood that the response signals can correspond to impedance values, and the impedance values can further correspond to determined reactance and resistance parameter values that comprise the determined impedance values.

FIG. 2 schematically illustrates an exemplary sequence for a single quadripolar bioimpedance measurement performed by the medical device 100 on the breasts 20. A first pair of bioimpedance electrodes 105 is designated as a current injection pair (I+ and I−), and a second, different pair of bioimpedance electrodes 105 is designated as a voltage sensing pair (V+ and V−). In operation, the analog front end 125 injects an alternating excitation current through the breast tissue between I+ and I−, establishing a sensitive field within a region of tissue, while the differential voltage resulting from the current flow is sensed between V+ and V−. From the known excitation current and the sensed differential voltage, the system computes the complex impedance Z(f)=R(f)+jX(f) at a selected frequency, where R(f) denotes resistance and X(f) denotes reactance, in some implementations.

In some implementations, the sequence diagram in FIG. 2 uses arrows labeled “Excitation current” and “Measured voltage” to depict the concurrent actions of driving current via the I+/I− pair and sensing voltage via the V+/V− pair, respectively. The indicated sensitive field in FIG. 2 represents the region of tissue most contributing to the measured impedance for the particular electrode geometry. The illustrated step corresponds to one measurement “beam” within a programmed series of quadripolar assignments that, in some implementations, iteratively varies which electrodes 105 act as I+/I− and which electrodes 105 act as V+/V− to sample different spatial paths through the breast tissue.

In some implementations, the sequence shown in FIG. 2 operates within the hardware context depicted in FIG. 1, where the bioimpedance electrodes 105, a multiplexer 120, the analog front end 125, and a microcontroller 130 cooperate to realize electronically reconfigurable quadripolar topologies. The multiplexer 120, under control of the microcontroller 130, dynamically routes the drive and sense terminals of the analog front end 125 to selected electrodes 105 to implement the I+/I− and V+/V− roles without physical movement of the medical device 100. The microcontroller 130, in some implementations, issues control signals to the multiplexer 120 to select the electrode-pair combination, configures the analog front end 125 for the target excitation, and acquires digitized measurements for local preprocessing and storage.

In some implementations, FIG. 2 is a simplified representation of electrode roles and signal flow rather than a literal depiction of all electrodes present on the medical device 100. While FIG. 2 illustrates a minimal ring to explain I+/I− and V+/V− roles, each cup of the device 100, as shown in FIG. 1, can implement a dense array of bioimpedance electrodes 105 arranged in multiple branches (e.g., twelve branches of five pads per branch) with up to four additional pads, for a total of 64 bioimpedance connection points per cup in some implementations. The multiplexer 120 can connect, under electronic control, the drive outputs and sense inputs of the analog front end 125 to any selected electrodes 105 so that, during an individual quadripolar read, only four pads (two drive and two sense) are active and the remaining pads are inactive. The system then steps through a programmed list of combinations to realize many beam geometries across the breast, in some implementations.

In some implementations, frequency selection is performed per beam. The microcontroller 130 instructs the analog front end 125 to inject a programmable alternating current at one or more discrete frequencies (e.g., approximately 4 kHz, 40 kHz, and 80 kHz; optionally including approximately 8 kHz, 18 kHz, and 128 kHz), thereby probing extracellular, membrane-associated, and intracellular pathways, respectively. Excitation amplitude is selected within a safe microcurrent range (e.g., approximately 8 μA to 100 μA) so that the sensed differential input remains within linear operating bounds. For each quadripolar step, in some implementations, the analog front end 125 demodulates the sensed voltage at the drive frequency and provides complex impedance samples at a configured output rate; digital high-pass and low-pass filters are applied to stabilize the quasi-static measurement, and multiple consecutive samples are averaged to produce a stabilized R and X for that geometry and frequency.

In some implementations, the microcontroller 130 performs quality validation before or during each read, including contact checks at the electrode-skin interface and compliance/over-range monitors, and rejects or flags measurements falling outside acceptance windows. In some implementations, the device 100 implements a bipolar contact indicator to assess electrode-skin interface quality prior to quadripolar or tripolar measurement steps. In some implementations, a bipolar contact impedance threshold is used as a gating criterion (e.g., a target Z_contact below approximately 1 MΩ), with visual or programmatic indicators prompting repositioning or pressure adjustment when the threshold is not met. In some implementations, contact indicators are monitored continuously or polled before each electrode reassignment in the programmed sequence, and only measurements satisfying the contact criterion are retained for analysis. The microcontroller 130 records timing metadata, the identities of the electrodes 105 assigned to I+/I− and V+/V− by the multiplexer 120, the selected frequency, and quality indicators to maintain traceability across the programmed sequence. Repeating the assignment of I+/I− and V+/V− across the array enables spatial sampling of the breast tissue volume without mechanically repositioning the medical device 100, and the resulting per-beam measurements can be used to compute parameters indicative of tissue condition and, in some implementations, to support reconstruction of two-dimensional slices and three-dimensional volumes.

In some implementations, the medical device 100 further supports a tripolar measurement configuration, additionally, or alternatively to the quadripolar measurement configuration, to enhance spatial resolution and contact assessment. In a tripolar configuration, a first electrode is designated as a common reference while current is injected between the reference and a selected electrode and the differential voltage is sensed between the reference and another selected electrode, thereby locally probing tissue properties in the vicinity of the reference electrode. In some implementations, tripolar measurements are executed as preliminary or interleaved steps within the programmed sequence to refine local mapping of contact impedance and to increase the density of spatial sampling without physically moving the device 100. In some implementations, the microcontroller 130 coordinates tripolar addressing through the multiplexer 120 and records tripolar results together with quadripolar results for downstream processing.

In some implementations, the temperature sensors 110 and PPG sensors 115 of FIG. 1 can be acquired in coordination with the quadripolar sequence to provide thermal and optical perfusion context. The PPG sensors 115 may be positioned to interrogate superficial vasculature and, in certain embodiments, an artery 25 that supplies the breast, while the temperature sensors 110 provide cutaneous temperature measurements that can be correlated with impedance-derived features. These additional data can be fused with the bioimpedance measurements to refine assessment of the physiological state of the breasts 20.

In summary, FIG. 2 portrays a single-step instance in which the microcontroller 130 addresses the multiplexer 120 to connect the analog front end 125 to designated electrodes 105 for I+/I− and V+/V−, the analog front end 125 drives an excitation at a programmed frequency and demodulates the sensed voltage, complex impedance samples are filtered and averaged to yield stabilized resistance and reactance for that beam and frequency, and the medical device 100 advances to the next programmed electrode-pair combination and, if applicable, the next frequency. In some implementations, this sequence is repeated across many combinations to generate a comprehensive set of bioimpedance measurements along different spatial paths through the breast tissue, enabling spatially resolved analysis without physical movement of the device 100.

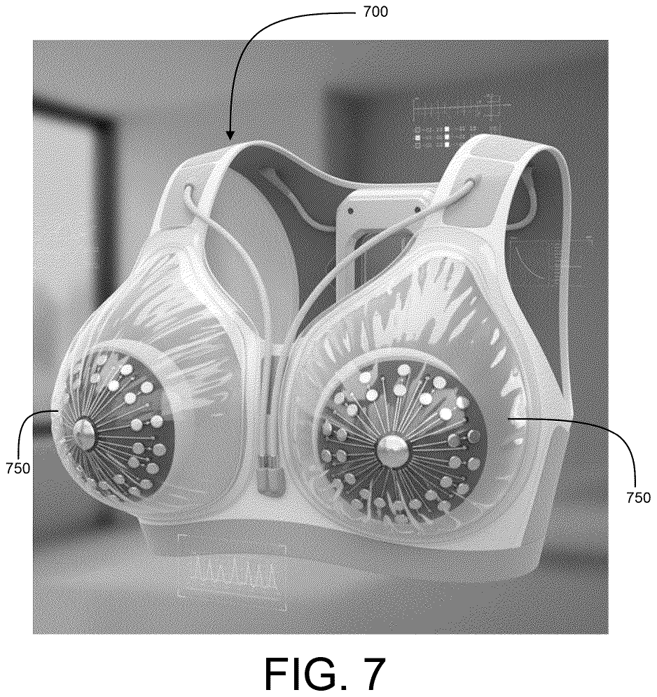

In some implementations, FIG. 3 illustrates a method 300 for assessing a condition of breast tissue of a subject using the medical device 100. In some implementations, prior to executing step 302, the method includes a fit-optimization step in which the microcontroller 130 inflates pneumatic support elements (e.g., as shown with respect to medical device 700, described below with reference to FIG. 7) to improve electrode-skin coupling. In some implementations, inflation proceeds under closed-loop control until a contact criterion is satisfied (e.g., a bipolar contact impedance below a predetermined threshold across the targeted electrodes and/or a target pneumatic pressure range), after which step 302 proceeds. In some implementations, if the contact criterion is not satisfied within a safety envelope (e.g., maximum inflation pressure or cycle count), the method prompts repositioning and reattempts fit optimization.

As shown, the method begins at step 302 by executing a programmed measurement sequence that defines a set of quadripolar electrode-pair combinations among bioimpedance electrodes 105 positioned over the breasts 20. In some implementations, executing the programmed measurement sequence comprises controlling a multiplexing network 120 to selectively route, under electronic control, drive terminals of an analog front end 125 to any selected electrodes 105 designated as a current injection pair (I+ and I−), and sense terminals of the analog front end 125 to any selected electrodes 105 designated as a voltage sensing pair (V+ and V−), thereby establishing, without physically moving the garment, a plurality of distinct quadripolar topologies across the breast surface. In some implementations, the programmed measurement sequence further specifies a set of discrete excitation frequencies, including a frequency sweep between approximately 4 kHz and approximately 128 kHz, and a sequence order (e.g., low-to-high) to probe extracellular, membrane-associated, and intracellular pathways. In some implementations, temperature-based calibration is performed using a neutral reference site on the body, such as a location on the back, to mitigate inter-breast asymmetries due to vascularization or hormonal state. In some implementations, the neutral calibration value is acquired contemporaneously with breast measurements and used to tare temperature-dependent terms and to reduce bias when computing parameters indicative of tissue condition.

In some implementations, at step 304 the method generates a set of bioimpedance measurements across different spatial paths through the breast tissue by iterating the quadripolar assignments defined in the programmed measurement sequence. For each quadripolar assignment, the analog front end 125 injects an alternating current between the selected I+ and I− electrodes 105 and senses a differential voltage between the selected V+ and V− electrodes 105, thereby producing complex impedance samples Z(f) comprising resistance R(f) and reactance X(f) at the selected frequency. In some implementations, the device 100 applies signal conditioning including, for example, contact validation, lead/compliance monitoring, and digital filtering to the complex impedance samples, and averages multiple consecutive samples per electrode-pair combination and per frequency to produce stabilized R and X values. In some implementations, the multiplexing network 120 steps through electrode-pair combinations that form sector-based and line-scan traversals, creating a plurality of beam geometries suitable for spatial sampling and, in some implementations, image reconstruction.

In some implementations, the method optionally acquires, in parallel or in defined phases, temperature data from temperature sensors 110 and optical pulse data from PPG sensors 115. The temperature data can provide spatially resolved cutaneous temperature measurements, and the PPG data can provide perfusion context. In some implementations, the microcontroller 130 coordinates data acquisition timing, frequency selection, and electrode addressing, records metadata identifying the I+/I− and V+/V− electrodes 105 used for each measurement, and stores descriptive statistics (e.g., mean, minimum, maximum, and standard deviation) for traceability and quality control. In some implementations, the device 100 stores, for each electrode-pair combination, a multi-frequency vector of resistance R(f) and reactance X(f) values corresponding to the programmed excitation frequencies, thereby preserving, per geometry, the R/X spectrum used for subsequent analysis and modeling. In some implementations, this per-geometry R/X spectrum is maintained together with acquisition metadata, including electrode identifiers for I+/I− and V+/V−, frequency indices, filter states, timestamps, and quality flags, to support downstream modeling, reconstruction, auditing, and reproducibility.

In some implementations, at step 306 the method determines, based on the resistance and reactance values, a parameter indicative of the breast tissue condition. The parameter, in some implementations, comprises one or more of: (i) a spatially resolved image of impedance-related values over a two-dimensional slice of a breast generated from the set of bioimpedance measurements (e.g., by Electrical Impedance Tomography using the stabilized R and/or X values at one or more frequencies), (ii) a three-dimensional reconstruction obtained by stacking a plurality of two-dimensional slices reconstructed at different anatomical levels and orientations, and (iii) a diagnostic score corresponding to presence or progression of a pathology. In some implementations, generating the spatially resolved image comprises solving an Electrical Impedance Tomography (EIT) inverse problem that reconstructs pixel values on the slice from stabilized resistance and/or reactance measurements acquired along distinct electrode-pair paths. In some implementations, determining the parameter comprises performing a frequency-domain analysis in which, for each electrode-pair combination, the complex impedance samples are converted into R and X at each of the programmed frequencies, and optionally deriving additional spectroscopic features including impedance magnitude |Z|, phase angle, and ratios or differences between low-frequency and high-frequency impedances. In some implementations, the method further includes fitting a dispersion model (e.g., a Cole-Cole model) to multi-frequency complex impedance data to estimate parameters such as extracellular resistance (Re), intracellular resistance (Ri), infinite-frequency resistance (Roo), a dispersion shape factor (a), a characteristic time constant or frequency (r or fc), and an effective membrane capacitance (Cm), and deriving one or more features such as a normalized resistance/reactance vector (BIVA), extracellular-to-intracellular resistance ratios, or spectroscopic contrast metrics.

In some implementations, once dispersion parameters are estimated, the device 100 optionally performs extrapolation to infer tissue impedance characteristics at unmeasured frequencies. In some implementations, the fitted dispersion model is evaluated at intermediate or extended frequency points to generate estimated resistance and reactance values R(f_est) and X(f_est), which can be used for feature derivation, spectroscopic contrasts, or to harmonize datasets acquired with different frequency subsets.

In some implementations, the method includes additional processing in which the one or more processors of the device 100 standardize or normalize features derived from stabilized R and X (and optionally temperature and PPG features) relative to a reference dataset or a patient-specific baseline, to generate normalized inputs for a classification model. In some implementations, the method generates model inputs for a logistic classification model comprising at least resistance and reactance at two or more frequencies and one or more dispersion parameters; the logistic model is configured to produce a probability or risk score of a pathology, and in some implementations applies a classification threshold to convert the risk score into a diagnostic output. In some implementations, the method stores, with each computed parameter, the acquisition settings (electrode addressing, frequency, filter selections), quality indicators, and timestamps to ensure auditability.

In some implementations, the method optionally fuses temperature features derived from temperature sensors 110 and optical perfusion indicators derived from PPG sensors 115 with impedance-derived features, to enhance robustness to physiological variability and to improve discrimination between tissue states. In some implementations, the method comprises re-measurement triggers based on quality criteria, including a bipolar contact impedance check and a quadripolar impedance range validity window, prior to accepting data for use in parameter determination. In some implementations, the method completes by outputting the parameter indicative of the breast tissue condition as a spatial map, a volumetric reconstruction, or a diagnostic score, and by communicating the results, along with associated metadata, to an external user device.

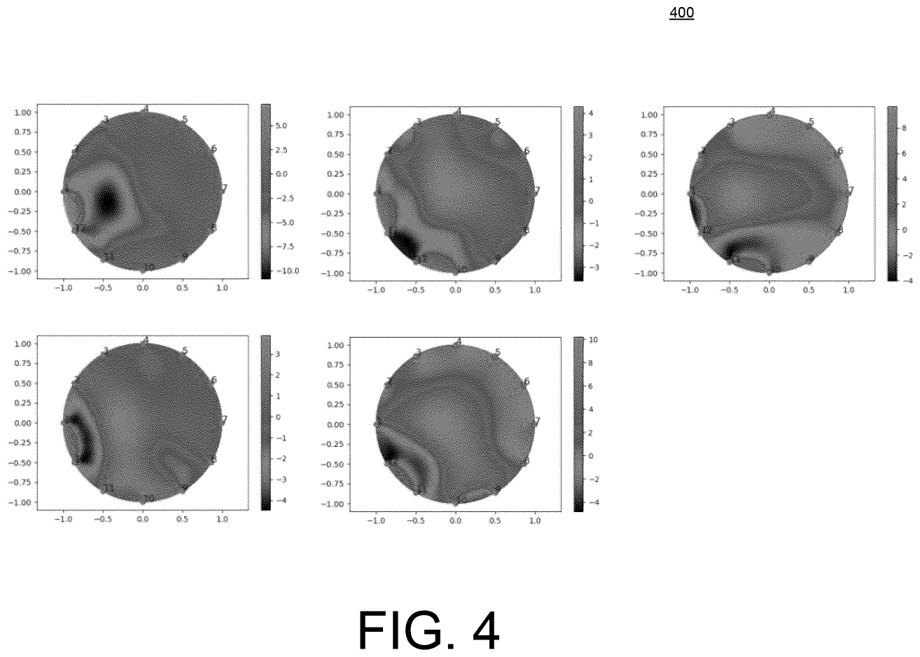

FIG. 4 shows example results produced by executing the foregoing method (e.g., method 300, described above with reference to FIG. 3) and indicates that a tumor is present. In some implementations, the displayed outputs include one or more of: a spatial parameter map with values that do not exceed a lesion-indicating threshold within the regions of interest; per-frequency impedance-based plots in which resistance and/or reactance features remain within reference bounds; and a classification output whose risk score is below a decision threshold, thereby yielding a “no tumor” determination. In some implementations, the “no tumor” indication is corroborated by the absence of extended regions exhibiting values consistent with lesion-associated patterns, and by concordant temperature or perfusion context where acquired. In some implementations, the method completes by outputting the parameter indicative of the breast tissue condition, storing the associated acquisition and quality metadata, and communicating the results to an external user device.

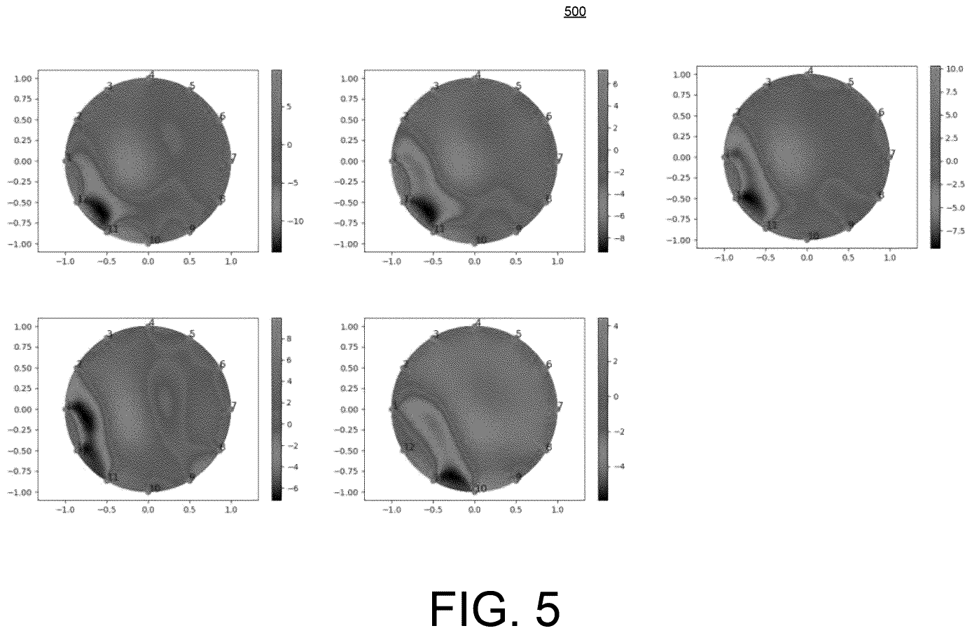

FIG. 5 shows example results produced by executing the foregoing method (e.g., method 300, described above with reference to FIG. 3) and indicates that no tumor is present. In some implementations, the displayed outputs include one or more of: (i) spatial maps in which values remain within reference bounds and do not exceed a lesion-indicating threshold within the regions of interest; (ii) per-frequency impedance-based plots in which the standardized resistance values are consistent with non-lesion patterns; and (iii) a classification result whose risk score is below a decision threshold. FIG. 5 contrasts with FIG. 4 by exhibiting a higher standardized resistance value (e.g., standardized R in FIG. 5 greater than standardized R in FIG. 4), which is consistent with the absence of extracellular fluid increase typically associated with inflammation; accordingly, the method yields a “no tumor” determination in FIG. 5 while FIG. 4 presents comparatively lower standardized resistance and a lesion-consistent pattern.

In some implementations, FIGS. 4-5 depict results rendered on a normalized resistance scale in which values can assume negative magnitudes due to statistical centering rather than indicating a physically negative resistance. In some implementations, the device 100 first acquires raw resistance values R(f) from quadripolar bioimpedance measurements at one or more frequencies using bioimpedance electrodes 105, the multiplexer 120, and the analog front end 125, as described herein. In some implementations, these raw R(f) values are then transformed into normalized or standardized quantities prior to visualization and decision-making, resulting in the negative scale shown in FIGS. 4-5.

In some implementations, normalization is performed by subtracting a reference value from the measured resistance and optionally dividing by a dispersion measure. In one non-limiting approach, a standardized resistance R*(f) is computed per beam, pixel, region, or breast as R*(f)=(R(f)−μref(f))/σref(f), where μref(f) and σref(f) are the mean and standard deviation determined from a reference set (e.g., a healthy population or a prior baseline established for the same subject). In some implementations, a mean-centered normalization is used without scaling, RA(f)=R(f)−Rref(f), where Rref(f) can be a contralateral-breast reference, a prior-session baseline for longitudinal follow-up, or a cohort-derived normative value. In some implementations, inter-breast asymmetry features are computed as left-right differences or ratios, which can also yield negative values when the measured breast is less resistive than the reference breast.

In some implementations, the normalized values (e.g., R*(f) or RA(f)) are mapped to a zero-referenced display in which zero represents the baseline (population or subject-specific), negative values indicate measurements below the baseline, and positive values indicate measurements above the baseline. In some implementations, this zero-referenced convention is preserved in spatial maps (e.g., two-dimensional slice images) and in region-wise plots, such that the color scale and axis tick labels include negative values even though the underlying physical resistance is non-negative. In some implementations, the device 100 stores with each normalized datum the identity of the underlying reference (e.g., cohort statistics, contralateral breast, or prior session) to ensure traceability.

In some implementations, FIG. 4 shows results rendered on a standardized R scale where certain regions may include negative values relative to the chosen baseline, and the aggregate spatial extent and magnitude of negative regions exceed a lesion-indicating threshold and the classification score exceeds a decision threshold, yielding an overall “tumor” determination. In some implementations, FIG. 5 likewise shows results on the same zero-referenced standardized scale, indicating “no tumor,” and may present higher standardized resistance values than FIG. 4 (e.g., standardized R in FIG. 5 greater than standardized R in FIG. 4) consistent with relatively lower extracellular fluid content and the absence of a lesion or tumor. Both FIG. 4 and FIG. 5 thus demonstrate that negative values on the R scale are a byproduct of normalization against a reference rather than an indication of physically negative resistance, and that diagnostic interpretation relies on thresholds, spatial extent, and model outputs derived from the normalized features.

In some implementations, normalization may be applied per frequency to create multi-frequency standardized vectors used for imaging and/or classification, and may be combined with filtering, averaging, and quality gating performed by the microcontroller 130 and the analog front end 125. In some implementations, the standardized R values are integrated with other features (e.g., reactance X(f), temperature from sensors 110, and, where available, optical perfusion context from PPG sensors 115) to generate parameters indicative of the condition of breast tissue, while the visualization layers preserve the normalized scale so that values below baseline appear as negative on the axes and color bars shown in FIGS. 4-5.

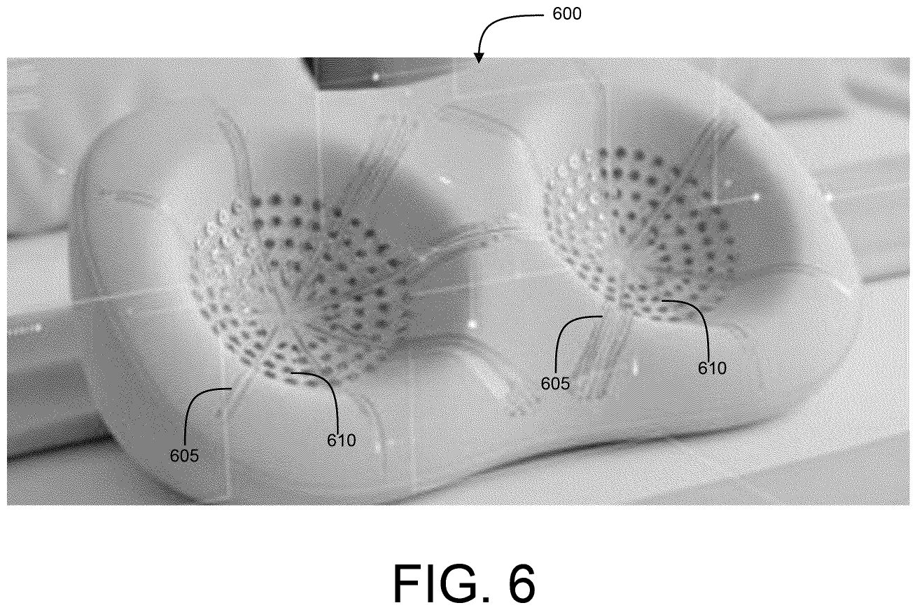

In some implementations, and as shown in FIG. 6, a medical device 600 is provided in the form of a cushion configured to receive and support the breasts 20 during measurement. In some implementations, medical device 600 includes bioimpedance electrodes 605, temperature sensors 610, and PPG sensors 615 (not shown) distributed across a contact surface to optimize electrode-skin interface regardless of breast morphology or patient position, and further includes a multiplexer 620 (not shown), an analog front end 625 (not shown), and a microcontroller 630 (not shown) for signal routing, acquisition, and control. In some implementations, medical device 600 also comprises an integrated region configured to receive an arm to facilitate adjunct measurements for lymphedema assessment and perfusion context (not shown in FIG. 6). In some implementations, medical device 600 is functionally similar to medical device 100 but differs in form factor, enabling stable contact under a variety of postures (e.g., supine or seated) and facilitating repeatable acquisition without garment adjustment. To avoid redundancy, elements and operation of medical device 600 that correspond to medical device 100 are omitted for brevity and are to be understood as analogous as labeled, with bioimpedance electrodes 605 being functionally similar to bioimpedance electrodes 105, temperature sensors 610 being functionally similar to temperature sensors 110, PPG sensors 615 being functionally similar to PPG sensors 115, multiplexer 620 being functionally similar to multiplexer 120, analog front end 625 being functionally similar to analog front end 125, and microcontroller 630 being functionally similar to microcontroller 130.

Although not shown in FIG. 6, in some implementations, medical device 600 includes additional temperature sensors 610 disposed to be in contact with a neutral reference site on the body, such as a location on the back of the user for temperature-based calibration and to mitigate inter-breast asymmetries due to vascularization or hormonal state. In some implementations, the neutral calibration value is acquired contemporaneously with breast measurements and used to tare temperature-dependent terms and to reduce bias when computing parameters indicative of tissue condition.

Referring to FIG. 7, in some implementations, a medical device 700 includes integrated pneumatic support elements 750 configured to optimize fit and electrode contact under varied breast morphologies and patient postures. In some implementations, medical device 700 includes bioimpedance electrodes functionally analogous to electrodes 105, temperature sensors analogous to sensors 110, PPG sensors analogous to sensors 115, a multiplexer functionally analogous to multiplexer 120, an analog front end functionally analogous to analog front end 125, and a microcontroller functionally analogous to microcontroller 130, and further comprises pneumatic support elements 750 disposed in the cups or bra structure. In some implementations, a pump and electronically actuated valves are coupled to the pneumatic support elements 750 and are commanded by the microcontroller 130 to inflate or deflate per a closed-loop policy using contact indicators (e.g., bipolar impedance and/or pneumatic pressure feedback) to achieve uniform and reproducible contact. In some implementations, the pneumatic feedback loop for pneumatic support elements 750 may be based on when Z_contact falls below a threshold (e.g., −1 MΩ) or based on pressure sensing of the pneumatic support elements 750, which may be inflated until the pressure threshold is met or a safety pressure limit is reached. In some implementations, the pneumatic support elements 750 are segmented to allow sector-specific pressure control aligned with sector-based or line-scan traversals, improving local contact in regions scheduled for quadripolar measurements. In some implementations, pressure, valve states, and contact metrics are stored with each acquisition to document contact quality and enhance reproducibility across sessions. In some implementations, the pneumatic support elements 750 are designed to accommodate and not obstruct optical paths associated with PPG sensors and to preserve airflow and skin comfort during prolonged use. In some implementations, once the target contact condition is achieved, the microcontroller maintains or gradually bleeds pressure to stabilize contact during acquisition, and records inflation pressure, valve states, and contact indicators as metadata associated with each measurement.

Although the example medical device described in the implementations herein are in reference to assessing breast tissue, it should be understood that the medical device is not so limited. In other implementations, the medical device can be configured to similarly assess other types of tissue, such as non-invasively determining levels of lung-inflammation and determining the likelihood of lung cancer.

The foregoing disclosure provides illustration and description but is not intended to be exhaustive or to limit the implementations to the precise form disclosed. Modifications may be made in light of the above disclosure or may be acquired from practice of the implementations. As used herein, the term “component” is intended to be broadly construed as hardware, firmware, or a combination of hardware and software. It will be apparent that systems and/or methods described herein may be implemented in different forms of hardware, firmware, and/or a combination of hardware and software. The actual specialized control hardware or software code used to implement these systems and/or methods is not limiting of the implementations. Thus, the operation and behavior of the systems and/or methods are described herein without reference to specific software code—it being understood that software and hardware can be used to implement the systems and/or methods based on the description herein. As used herein, satisfying a threshold may, depending on the context, refer to a value being greater than the threshold, greater than or equal to the threshold, less than the threshold, less than or equal to the threshold, equal to the threshold, and/or the like, depending on the context. Although particular combinations of features are recited in the claims and/or disclosed in the specification, these combinations are not intended to limit the disclosure of various implementations. In fact, many of these features may be combined in ways not specifically recited in the claims and/or disclosed in the specification.

Although each dependent claim listed below may directly depend on only one claim, the disclosure of various implementations includes each dependent claim in combination with every other claim in the claim set. No element, act, or instruction used herein should be construed as critical or essential unless explicitly described as such. Also, as used herein, the articles “a” and “an” are intended to include one or more items and may be used interchangeably with “one or more.” Further, as used herein, the article “the” is intended to include one or more items referenced in connection with the article “the” and may be used interchangeably with “the one or more.” Furthermore, as used herein, the term “set” is intended to include one or more items (e.g., related items, unrelated items, a combination of related and unrelated items, and/or the like), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”).

What is claimed is:

1. A wearable medical device for assessing a condition of breast tissue of a subject, comprising:
a garment configured to position a first sensor array over a first breast and a second sensor array over a second breast, each sensor array including a plurality of bioimpedance electrodes arranged on a flexible substrate and configured to contact skin of the corresponding breast;
an analog front-end configured to perform quadripolar bioimpedance measurements by injecting an alternating current into the breast tissue via a selected pair of the bioimpedance electrodes and sensing a differential voltage via a different selected pair of the bioimpedance electrodes to obtain complex impedance samples comprising resistance and reactance values at one or more frequencies;
a multiplexing network operatively coupled between the analog front-end and the plurality of bioimpedance electrodes and configured to selectively route, under electronic control, drive terminals of the analog front-end to any electrodes of the plurality of bioimpedance electrodes for current injection, and sense terminals of the analog front-end to any electrodes of the plurality of bioimpedance electrodes for voltage measurement, thereby implementing a plurality of distinct electrode-pair combinations;
one or more processors and a non-transitory memory storing instructions that, when executed, cause the device to:
control the multiplexing network to execute a programmed measurement sequence defining a set of quadripolar electrode-pair combinations to generate a set of bioimpedance measurements across different spatial paths through the breast tissue; and
determine, based on the resistance and reactance values, a parameter indicative of the breast tissue condition.

2. The device of claim 1, wherein the parameter comprises one or more of (i) a spatially resolved image of impedance-related values over a two-dimensional slice of the breast generated from the set of bioimpedance measurements, (ii) a three-dimensional reconstruction obtained from a plurality of two-dimensional slices, and (iii) a diagnostic score corresponding to presence or progression of a pathology.

3. The device of claim 1, wherein the sensor array further comprises a plurality of temperature sensors configured to measure cutaneous temperature at multiple locations on the corresponding breast and the memory stores instructions, that when executed, cause the device to:
acquire temperature data from the plurality of temperature sensors; and
determine the parameter further based on the acquired temperature data.

4. The device of claim 1, wherein the sensor array further comprises one or more photoplethysmography (PPG) sensors, and the memory stores instructions, that when executed, cause the device to:
acquire PPG data from the one or more PPG sensors; and
determine the parameter further based on the acquired PPG data.

5. The device of claim 1, wherein the programmed measurement sequence comprises two or more frequencies.

6. The device of claim 1, wherein the programmed measurement sequence comprises a frequency sweep between approximately 4 kHz and approximately 128 kHz.

7. The device of claim 1, wherein implementing the plurality of distinct electrode-pair combinations is performed without physically moving the garment.

8. The device of claim 1, wherein the memory stores instructions, that when executed, cause the device to:
perform signal conditioning on the bioimpedance measurements including at least contact validation and averaging to produce stabilized resistance and reactance values per electrode-pair combination and per frequency;
wherein the parameter indicative of the breast tissue condition is based on the stabilized resistance and reactance values.

9. The device of claim 1, wherein the one or more processors are further configured to perform a frequency-domain analysis comprising:
for each of a plurality of discrete excitation frequencies within a sweep spanning from approximately 4 kHz to approximately 128 kHz, driving an alternating current through a selected drive electrode pair and demodulating a corresponding sensed voltage at a selected sense electrode pair to obtain complex impedance samples;
converting the complex impedance samples into resistance (R) and reactance (X) values expressed in ohms for each frequency; and
storing, for each electrode-pair combination, a multi-frequency vector of R and X values usable as inputs to downstream modeling.

10. The device of claim 9, wherein the frequency-domain analysis further comprises calculating, per frequency and per electrode-pair combination, one or more of (i) impedance magnitude |Z|, (ii) impedance phase angle, and (iii) ratios or differences between low-frequency and high-frequency impedances, including a ratio of Z at a low frequency to Z at a high frequency.

11. The device of claim 1, wherein the one or more processors are further configured to fit a Cole-Cole model to the multi-frequency complex impedance data to estimate dispersion parameters comprising one or more of (i) extracellular resistance (Re), (ii) intracellular resistance (Ri), (iii) infinite-frequency resistance (Roo), (iv) a dispersion shape factor (a), (v) a characteristic time constant or frequency (r or fc), and an effective membrane capacitance (Cm).

12. The device of claim 11, wherein the one or more processors are configured to derive, from the fitted Cole-Cole model and the measured data, one or more features selected from: (i) phase angle at a specified frequency, (ii) a vector composed of normalized resistance and reactance (BIVA), (iii) an extracellular-to-intracellular resistance ratio, and (iv) at least one spectroscopic contrast metric formed from two or more frequencies.

13. The device of claim 1, wherein the one or more processors are configured to generate model inputs for a logistic classification model, the model inputs comprising one or more of (i) resistance and reactance values, (ii) one or more Cole-Cole parameters, and (iii) temperature-derived metrics.

14. The device of claim 13, wherein the logistic classification model comprises a binary logistic regression configured to output a probability or risk score of a pathology, the binary logistic regression receiving as explanatory variables one or more of (i) resistance at approximately 80 kHz, (ii) resistance and reactance at approximately 40 kHz, (iii) resistance and reactance at approximately 4 kHz, and (iv) temperature-derived features.

15. The device of claim 13, wherein the one or more processors are configured to standardize at least a subset of the model inputs by mean-centering or dividing by a standard deviation derived from a reference dataset or a patient-specific baseline to generate normalized features prior to logistic classification.

16. The device of claim 9, wherein converting complex impedance samples into resistance and reactance values comprises:
demodulating the sensed voltage at the excitation frequency;
applying programmable bioimpedance-channel high-pass filters, low-pass filters, or both high-pass and low-pass filters; and
averaging a plurality of successive samples to obtain stabilized R and X per frequency.

17. The device of claim 1, wherein the one or more processors are configured to reject bioimpedance data that fails a quality criterion including one or more of (i) a bipolar contact impedance threshold, (ii) a quadripolar impedance range validity window, (iii) a current-generator compliance monitor threshold, and (iv) a digital over-/under-voltage lead-off threshold.

18. The device of claim 1, wherein the multiplexing network is configured to execute programmed sequences defining sector-based traversals and line-scan traversals, each traversal comprising a plurality of distinct electrode-pair combinations that create different sensitive current paths through the breast tissue, thereby producing beam data suitable for image reconstruction.

19. The device of claim 2, wherein generating the spatially resolved image over a two-dimensional slice comprises solving an Electrical Impedance Tomography (EIT) inverse problem.

20. The device of claim 2, wherein generating the three-dimensional reconstruction comprises stacking a plurality of two-dimensional slices reconstructed at different anatomical levels and orientations and interpolating between the levels to form a volumetric impedance map.
